(19) United States
(12) Patent Application Publication
(10) Pub. No.: US 2025/0281753 A1
(43) Pub. Date: Sep. 11, 2025

(54) CLOSED-LOOP FEEDBACK AND TREATMENT

(71) Applicant: Medtronic, Inc., Minneapolis, MN (US)

(72) Inventors: Maneesh Shrivastav, Blaine, MN (US); ShaileshKumar V. Musley, Brooklyn Park, MN (US); Kanthaiah Koka, Valencia, CA (US); Steven M. Goetz, North Oaks, MN (US); Suryakiran Vadrevu, Santa Clarita, CA (US); Rebecca K. Gottlieb, Culver City, CA (US); David John Miller, Austin, TX (US); Leonid M. Litvak, Los Angeles, CA (US)

(21) Appl. No.: 18/862,817

(22) PCT Filed: Apr. 7, 2023
(86) PCT No.: PCT/US2023/065510

§ 371 Date: Nov. 4, 2024

Related U.S. Application Data
(60) Provisional application No. 63/342,998, filed on May 17, 2022.

(51) Int. Cl. A61N 1/36

(52) U.S. Cl. CPC A61N 1/36139; A61N 1/36053; A61N 1/36171

(57) ABSTRACT

An illustrative system is described to include: a first sensor that measures a glycemic level of a patient; a second sensor that measures a protein level of the patient, a hormone level of the patient, and/or an activity level of the patient; a processor that receives inputs from the first sensor and inputs from the second sensor; and memory including data that, when executed by the processor, enables the processor to perform one or more functions. The one or more functions may include: analyzing the inputs received from the first sensor and the second sensor; determining, based on the analysis, that an electrical treatment is to be applied to the patient, where the electrical treatment includes application of at least one electrical signal to a nervous system of the patient; and causing the electrical treatment to be applied to the nervous system of the patient.

### CROSS REFERENCE TO RELATED APPLICATIONS

This application claims the benefit of U.S. Provisional Application No. 63/342,998, filed on May 17, 2022, entitled “Closed-Loop Feedback and Treatment”, and further identified as Attorney Docket No. A0008258US01 (10259-211-7P); U.S. Provisional Application No. 63/338,794, filed on May 5, 2022, entitled “Systems and Methods for Stimulating an Anatomical Element Using an Electrode Device”, and further identified as Attorney Docket No. A0008247US01 (10259-211-1P); U.S. Provisional Application No. 63/339,049, filed on May 6, 2022, entitled “Systems and Methods for Mechanically Blocking a Nerve”, and further identified as Attorney Docket No. A0008250US01 (10259-211-2P); U.S. Provisional Application No. 63/338,806, filed on May 5, 2022, entitled “Systems and Methods for Wirelessly Stimulating or Blocking at Least One Nerve”, and further identified as Attorney Docket No. A0008251US01 (10259-211-3P); U.S. Provisional Application No. 63/339,101, filed on May 6, 2022, entitled “Neuromodulation Techniques to Create a Nerve Blockage with a Combination Stimulation/Block Therapy for Glycemic Control”, and further identified as Attorney Docket No. A0008252US01 (10259-211-4P); U.S. Provisional Application No. 63/339,136, filed on May 6, 2022, entitled “Neuromodulation for Treatment of Neonatal Chronic Hyperinsulinism”, and further identified as Attorney Docket No. A0008253US01(10259-211-5P); U.S. Provisional Application No. 63/342,945, filed on May 17, 2022, entitled “Neuromodulation Techniques for Treatment of Hypoglycemia”, and further identified as Attorney Docket No. A0008255US01 (10259-211-6P); U.S. Provisional Application No. 63/338,817, filed on May 5, 2022, entitled “Systems and Methods for Monitoring and Controlling an Implantable Pulse Generator”, and further identified as Attorney Docket No. A0008259US01 (10259-211-8P); U.S. Provisional Application No. 63/339,024, filed on May 6, 2022, entitled “Programming and Calibration of Closed-Loop Vagal Nerve Stimulation Device”, and further identified as Attorney Docket No. A0008260US01 (10259-211-9P); U.S. Provisional Application No. 63/339,304, filed on May 6, 2022, entitled “Systems and Methods for Stimulating or Blocking a Nerve Using an Electrode Device with a Sutureless Closure”, and further identified as Attorney Docket No. A0008262US01 (10259-211-11P); U.S. Provisional Application No. 63/339,154, filed on May 6, 2022, entitled “Personalized Machine Learning Algorithm for Stimulation/Block Therapy for Treatment of Type 2 Diabetes”, and further identified as Attorney Docket No. A0008263US01 (10259-211-12P); U.S. Provisional Application No. 63/342,967, filed on May 17, 2022, entitled “Patient User Interface for a Stimulation/Block Therapy for Treatment of Type 2 Diabetes”, and further identified as Attorney Docket No. A0008264US01 (10259-211-13P); and U.S. Provisional Application No. 63/339,160, filed on May 6, 2022, entitled “Utilization of Growth Curves for Optimization of Type 2 Diabetes Treatment”, and further identified as Attorney Docket No. A0008265US02 (10259-211-14P), all of which applications are incorporated herein by reference in their entireties.

### BACKGROUND

The present disclosure is generally directed to therapeutic neuromodulation and relates more particularly to a stimulation/block therapy to affect glycemic control of a patient.

Diabetes represents a large and growing global health issue with estimates of over 400 million patients worldwide having been diagnosed with type 2 diabetes and estimates of 4.2 million annual deaths related to complications of diabetes. Despite different types of treatments being developed and utilized (e.g., medication, surgery, diet, etc.), type 2 diabetes remains challenging to effectively treat. Type 2 patients must frequently contend with keeping their blood sugar levels in a desirable glycemic range. Prolonged deviations can lead to long term complications such as retinopathy or kidney damage. Because treatment for diabetes is self-managed by the patient on a day-to-day basis (e.g., the patients self-inject the insulin), compliance or adherence with treatments can be problematic.

### BRIEF SUMMARY

Example aspects of the present disclosure include:

A system, including: a first sensor that measures a glycemic level of a patient; a second sensor that measures at least one of a protein level of the patient, a hormone level of the patient, and an activity level of the patient; a processor that receives inputs from the first sensor and inputs from the second sensor; and memory including data that, when executed by the processor, enables the processor to: analyze the inputs received from the first sensor and the second sensor; determine, based on the analysis, that an electrical treatment is to be applied to the patient, where the electrical treatment includes application of at least one electrical signal to a nervous system of the patient; and cause the electrical treatment to be applied to the nervous system of the patient.

In some embodiments, the electrical treatment includes application of a first electrical signal to a first portion of a nerve and application of a second electrical signal to a second portion of the nerve.

In some embodiments, the first electrical signal includes a low frequency stimulation of a celiac branch of the nerve and the second electrical signal includes a high frequency blockade of a hepatic branch of the nerve.

In some embodiments, the first electrical signal includes a frequency of no more than about 5 kHz.

In some embodiments, the second electrical signal includes a square wave having a frequency of between about 1-10 Hz.

In some embodiments, the second electrical signal includes an ultralow frequency blockade of a hepatic branch of the nerve.

In some embodiments, the second sensor measures the protein level of the patient, the system further includes: a third sensor that measures the hormone level of the patient.

In some embodiments, the memory further includes data that, when executed by the processor, enables the processor to analyze the inputs received from the first sensor, the second sensor, and the third sensor and further enables the processor to determine, based on the analysis, that the electrical treatment is to be applied to the patient.

In some embodiments, the electrical treatment is applied when the following conditions are met: (i) the measured glycemic level exceeds a predetermined glycemic threshold OR (ii) the measured protein level exceeds a predetermined protein threshold AND the measured hormone level exceeds a predetermined hormone threshold.

In some embodiments, the electrical treatment is applied when the measured activity level of the patient is below a predetermined activity threshold.

In some embodiments, the second sensor includes an activity sensor that measures the activity level of the patient.

In some embodiments, the activity sensor includes at least one of a heart rate sensor, an accelerometer, a gyroscope, and a motion sensor.

In some embodiments, first sensor includes a continuous glucose monitor.

In another example, a device is disclosed to include: a processor that receives a first input from a first sensor and a second input from a second sensor, where the first input describes a glycemic level of a patient, and where the second input describes at least one of a hormone level, a protein level, and an activity level of the patient; and memory including data that, when executed by the processor, enables the processor to; analyze the first input and the second input; determine, based on the analysis, that a treatment is to be applied to the patient, where the treatment includes application of at least one electrical signal to a nervous system of the patient; and cause the treatment to be applied to the patient.

In some embodiments, the treatment includes application of a first electrical signal to a first portion of a nerve and application of a second electrical signal to a second portion of the nerve, where first electrical signal comprises a low frequency stimulation of a celiac branch of the nerve, and where the second electrical signal includes a high frequency blockade of a hepatic branch of the nerve.

In some embodiments, the processor receives a third input from a third sensor, where the second input describes the hormone level, where the third input describes the protein level, and where the memory further includes data that, when executed by the processor, enables the processor to analyze the third input along with the first input and the second input, then determine, based on the analysis, that the treatment is to be applied to the patient.

In some embodiments, the processor receives a fourth input from a fourth sensor, where the fourth input describes the activity level, and where the memory further includes data that, when executed by the processor, enables the processor to analyze the fourth input along with the first input, the second input, and the third input, then determine, based on the analysis, that the treatment is to be applied to the patient.

In some embodiments, the fourth sensor includes at least one of a heart rate sensor, an accelerometer, a gyroscope, and a motion sensor.

In another example, a closed-loop system for providing therapy to a patient is provided, the system including: a plurality of sensors, where the plurality of sensors measure two or more of a glycemic level of the patient, a hormone level of the patient, a protein level of the patient, and an activity level of the patient; a processor; and memory including data that, when executed by the processor, enables the processor to; receive inputs from the plurality of sensors; analyze the inputs received from the plurality of sensors; determine, based on the analysis, that a treatment is to be applied to the patient, where the treatment includes application of at least one electrical signal to a nervous system of the patient; and cause the treatment to be applied to the patient.

In some embodiments, the system further includes: a first electrode that delivers a first electrical signal to a first portion of a nerve; and a second electrode that delivers a second electrical signal to a second portion of the nerve.

In some embodiments, the processor and the memory are included in an implantable pulse generator.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a diagram of a system according to at least one embodiment of the present disclosure;

FIG. 2 is a diagram of a first electrode and a second electrode according to at least one embodiment of the present disclosure;

FIG. 3 is a flow diagram illustrating a method according to at least one embodiment of the present disclosure;

FIG. 4 is a block diagram of a system according to at least one embodiment of the present disclosure; and

FIG. 5 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., random-access memory (RAM), read-only memory (ROM), electrically erasable programmable ROM (EEPROM), flash memory, or any other medium that can be used to store desired program code in the form of instructions or data structures and that can be accessed by a computer). The processors listed herein are not intended to be an exhaustive list of all possible processors that can be used for implementation of the described techniques, and any future iterations of such chips, technologies, or processors may be used to implement the techniques and embodiments of the present disclosure as described herein.

Instructions may be executed by one or more processors, such as one or more digital signal processors (DSPs), general purpose microprocessors (e.g., Intel Core i3, i5, i7, or i7 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; ARM, M0, M3, or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 4000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

Vagus nerve stimulation (VNS) is a technology that has been developed to treat different disorders or ailments of a patient, such as epilepsy and depression. In some examples, VNS involves placing a device in or on a patient's body that uses electrical impulses to stimulate the vagus nerve. For example, the device may be usually placed under the skin of the patient, where a wire (e.g., lead) and/or electrode connects the device to the vagus nerve. Once the device is activated, the device sends signals through the vagus nerve to the patient's brainstem (e.g., or different target area in the patient, such as other organs of the patient), transmitting information to their brain. For example, with VNS, the device may be configured to send regular, mild pulses of electrical energy to the brain via the vagus nerve. In some examples, this device may be referred to as an implantable pulse generator. An implantable vagus nerve stimulator has been approved to treat epilepsy and depression in qualifying patients.

The vagus nerve (e.g., also called the pneumogastric nerve, vagal nerve, the cranial nerve X, etc.) is responsible for various internal organ functions of a patient, including digestion, heart rate, breathing, cardiovascular activity, and reflex actions (e.g., coughing, sneezing, swallowing, and vomiting). Most patients may have one vagus nerve on each side of their body, with numerous branches running from their brainstem through their neck, chest, and abdomen down to part of their colon. The vagus nerve plays a role in many bodily functions and may form a link between different areas of the patient, such as the brain and the gut. The vagus nerve is a critical nerve for supplying parasympathetic information to the visceral organs of the respiratory, digestive, and urinary systems. Additionally, the vagus nerve is important in the control of heart rate, bronchoconstriction, and digestive processes. In some cases, the vagus nerve may be considered a mixed nerve based on including both afferent (sensory) fibers and efferent (motor) fibers. As such, based on including the two types of fibers, the vagus nerve may be responsible for carrying motor signals to organs for innervating the organs (e.g., via the efferent fibers), as well as carrying sensory information from the organs back to the brain (e.g., via the afferent fibers).

The vagus nerve has a number of different functions. Four key functions of the vagus nerve are carrying sensory signals, carrying special sensory signals, providing motor functions, and assisting in parasympathetic functions. For example, the sensory signals carried by the vagus nerve may include signaling between the brain and the throat, heart, lungs, and abdomen. The special sensory signals carried by the vagus nerve may provide signaling of special senses in the patient, such as the taste sensation behind the tongue. Additionally, the vagus nerve may enable certain motor functions of the patient, such as providing movement functions for muscles in the neck responsible for swallowing and speech. The parasympathetic functions provided by the vagus nerve may include digestive tract, respiration, and heart rate functioning. In some cases, the nervous system can be divided into two areas: sympathetic and parasympathetic. The sympathetic side increases alertness, energy, blood pressure, heart rate, and breathing rate. The parasympathetic side, which the vagus nerve is heavily involved in, decreases alertness, blood pressure, and heart rate, and helps with calmness, relaxation, and digestion.

VNS is considered a type of neuromodulation (e.g., a technology that acts directly upon nerves of a patient, such as the alteration, or “modulation,” of nerve activity by delivering electrical impulses or pharmaceutical agents directly to a target area). For example, as described above, VNS may include using a device (e.g., implanted in a patient or attached to the patient) that is configured to send regular, mild pulses of electrical energy to a target area of the patient (e.g., brainstem, organ, etc.) via the vagus nerve. Electrical pulses or impulses of this nature may affect how that target area of the patient functions to potentially treat different disorders or ailments of a patient. This may occur by either increasing the function of an organ or system by exciting or stimulating the necessary pathways, or by decreasing the function by blocking normal nerve activation to that system.

In some examples, for epileptic patients that suffer from seizures, VNS may change how brain cells work by applying electrical stimulation to certain areas involved in seizures. For example, research has shown that VNS may help control seizures by increasing blood flow in key areas, raising levels of some brain substances (e.g., neurotransmitters) important to control seizures, changing electroencephalogram (EEG) patterns during a seizure, etc. As an example, an epileptic patient's heart rate may increase during a seizure or epileptic episode, so the VNS device may be programmed to send stimulation to the vagus nerve regular intervals and when periods of increased heart rate are seen, where applying stimulation at those times of increased heart rate may help stop seizures. Additionally or alternatively, depression has been tied to an imbalance in certain brain chemicals (e.g., neurotransmitters), so VNS is believed to assist in treating patients diagnosed with depression by using electricity (e.g., electrical pulses/impulses) to influence the production of those brain chemicals.

Diabetes represents a large and growing global health issue with estimates of over 537 million patients worldwide having been diagnosed with type 2 diabetes and estimates of 6.7 million annual deaths related to complications of diabetes. Despite different types of treatments being developed and utilized (e.g., medication, surgery, diet, etc.), type 2 diabetes remains challenging to effectively treat. Type 2 patients must frequently contend with keeping their blood sugar levels in a desirable glycemic range. Prolonged deviations can lead to long term complications such as retinopathy or kidney damage. Because treatment for diabetes is self-managed by the patient on a day-to-day basis (e.g., the patients self-inject the insulin), compliance or adherence with treatments can be problematic. Additionally, in a financial sense, global expenditures for type 2 diabetes treatments, preventive measures, and resulting consequences are estimated at about $966 billion per year. Compounding this issue of high global expenditures is the increasing price of insulin.

As described herein, a neuromodulation technique is provided for glycemic control (e.g., as a treatment for diabetes) using a stimulation/block therapy (e.g., type of VNS). For example, this neuromodulation technique may generally include using a device (e.g., including at least an implantable pulse generator) to provide electrical stimulation (e.g., electrical pulses/impulses) on one or more trunks of the vagus nerve (e.g., vagal trunks) to mute a glycemic response for patients with diabetes. The “patient” as used herein may refer to homo sapiens or any other living being that has a vagus nerve.

In some examples, the device may provide stimulation/blocking of the celiac and hepatic vagal trunks (e.g., using the device) for the purposes of glycemic control. For example, the anterior sub diaphragmatic vagal trunk at the hepatic branching point of the vagus nerve may be electrically blocked (e.g., down-regulated) by delivering a high frequency stimulation (e.g., of about 5 kilohertz (kHz) or in a range between 1 kHz to 50 kHz). Other waveforms, such as ultralow frequency, long pulsewidth DC-like stimulation patterns may alternatively be used for such blocking. This has the effect of reducing the liver's output of glucose at a time when levels are already high. Type 2 patients must frequently contend with keeping their blood sugar levels in a desirable glycemic range. Prolonged deviations can lead to long term complications such as retinopathy or kidney damage. Additionally or alternatively, the posterior sub diaphragmatic vagal trunk at the celiac branching point of the vagus nerve may be electrically stimulated (e.g., up-regulated) by delivering a low frequency stimulation (e.g., a square wave at approximately 1 Hz or between 1 Hz and 10 Hz). Other waveforms, such as bursts of stimulation or non-square stimulation pulses may also be used to activate or electrically stimulation this pathway. Type 2 patients must frequently contend with keeping their blood sugar levels in a desirable glycemic range. Prolonged deviations can lead to long term complications such as retinopathy or kidney damage. In some examples, the electrical blocking and/or electrical stimulating of the respective vagal trunks may be performed by using one or more cuff electrodes (e.g., of the device) placed on the corresponding vagal trunks (e.g., sutured or otherwise held in place). The desired response by providing this stimulation/block therapy is a muting of the glycemic response of a patient. In some embodiments, cuff electrodes for stimulation may be placed at the posterior sub-diaphragmatic vagal trunk at the celiac branching point and sutured in place to deliver a square wave at approximately 1 Hz. The desired response is a muting of the glycemic response when the stimulation/block therapy is applied. In some examples, muting may refer to a lower post prandial peak of the glycemic response as compared to a peak without the stimulation/block therapy being applied. Additionally, therapy helps in bringing the post-prandial glucose excursions to more manageable levels faster than without stimulation/block therapy. In other words, not only does the therapy help reduce the peak but it also helps quickly return the peak down to baseline.

This concept is advantageous for those with type 2 diabetes where the postprandial glycemic response (e.g., occurring after a meal) can be very high. For example, some patients with type 2 diabetes may have high blood sugar levels (e.g., glucose levels) after eating a meal based on their reduced or lack of insulin production (e.g., normal insulin production in the body lowers blood sugar levels postprandially by promoting absorption of glucose from the blood into different cells). Additionally or alternatively, patients diagnosed with type 2 diabetes may generally have high glycemic levels at different points of the day (e.g., not necessarily postprandially or immediately after a meal). Over time, the effect of high glycemic values can have a detrimental effect on one's health, leading to neuropathy, retinopathy, and other ailments. Accordingly, by using the stimulation/block therapy provided herein, this high glycemic response experienced by type 2 diabetes patients may be muted (e.g., the glycemic response is reduced, particularly post prandially). Additionally, this therapy aims to improve insulin sensitivity by blocking hepatic glucose production and also by stimulating pancreatic insulin production needed for glycemic control, where the lack of insulin sensitivity can potentially lead to an imbalance in glycemic control and consequent systemic complications in patients with type 2 diabetes. In some examples, the therapy may also improve fasting hyperglycemia, which can be commonly seen in patients with type 2 diabetes.

As previously described, the electrical blocking and/or stimulation may be provided using electrodes. However, conventional electrode designs may use wires, which may be cumbersome and difficult to maintain. Thus, embodiments of the present disclosure provide technical solutions to one or more of the problems of (1) providing stimulation and/or blocking therapy, (2) automatically controlling or triggering stimulation/blocking therapies, and (3) increasing patient comfort and safety.

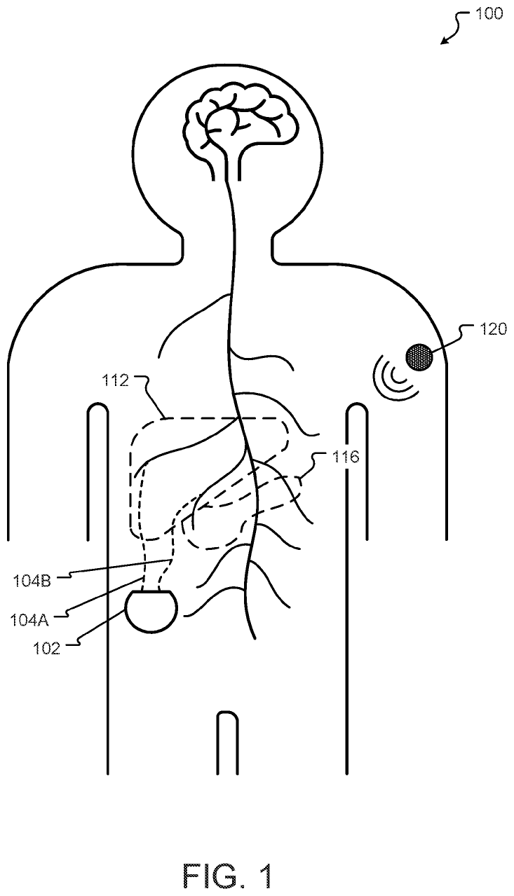

Turning to FIG. 1, a diagram of a system 100 according to at least one embodiment of the present disclosure is shown. The system 100 may be used to provide glycemic control for a patient and/or carry out one or more other aspects of one or more of the methods disclosed herein. For example, the system 100 may include a device 102 that is capable of providing a stimulation/blocking therapy that mutes a glycemic response for patients with diabetes. In some examples, the device 102 may be referred to as an implantable pulse generator, an implantable neurostimulator, or any other suitable type of device not explicitly listed or described herein. More specifically, the implantable pulse generator may be configured to generate a current or electrical signal. Additionally, the system 100 may include one or more wires 104 (e.g., leads) that provide a connection between the device 102 and nerves of the patient for enabling the stimulation/blocking therapy.

As described previously, neuromodulation techniques (e.g., technologies that act directly upon nerves of a patient, such as the alteration, or “modulation,” of nerve activity by delivering electrical impulses or localized pharmaceutical agents directly to a target area) may be used for assisting in treatments for different diseases, disorders, or ailments of a patient, such as epilepsy and depression. Accordingly, as described herein, these neuromodulation techniques may be used for muting a glycemic response in the patient to assist in the treatment of diabetes for the patient. For example, the device 102 may provide electrical stimulation to one or more trunks of the vagus nerve of the patient (e.g., via the one or more wires 104) to provide the stimulation/blocking therapy for supporting glycemic control in the patient.

In some examples, the one or more wires 104 may include at least a first wire 104A and a second wire 104B connected to respective vagal trunks (e.g., different trunks of the vagus nerve). As described previously, most patients have one vagus nerve on each side of their body, running from their brainstem through their neck, chest, and abdomen down to part of their colon. The vagus nerve plays a role in many bodily functions and may form a link between different areas of the patient, such as the brain and the gut. For example, the vagus nerve is responsible for various internal organ functions of a patient, including digestion, heart rate, breathing, cardiovascular activity, and reflex actions (e.g., coughing, sneezing, swallowing, and vomiting).

Accordingly, the first wire 104A may be connected to a first vagal trunk of the patient (e.g., the anterior sub diaphragmatic vagal trunk at the hepatic branching point of the vagus nerve) to provide an electrical blocking signal (e.g., a down-regulating signal) from the device 102 to that first vagal trunk (e.g., by delivering a high frequency stimulation, such as a high frequency signal having a frequency of about 5 kHz). Additionally or alternatively, the second wire 104B may be connected to a second vagal trunk of the patient (e.g., the posterior sub diaphragmatic vagal trunk at the celiac branching point of the vagus nerve) to provide an electrical stimulation signal (e.g., an up-regulating signal) from the device 102 to that second vagal trunk (e.g., by delivering a low frequency stimulation, such as a square wave or other waveform at 1 Hz). By providing this electrical blocking signal and the electrical stimulation signal to the respective vagal trunks, the system 100 may provide a muting of the glycemic response of the patient when the stimulation/blocking therapy is applied. For example, muting of the glycemic response may refer to a lower post prandial peak of the glycemic response as compared to a peak without the stimulation/block therapy being applied.

In some examples, the vagal trunks to which the wires 104 are connected may be connected to or otherwise in the vicinity of one or more organs of the patient, such that the blocking/stimulation signals provided to the respective vagal trunks by the wires 104 and the device 102 are delivered to the one or more organs. For example, the first vagal trunk (e.g., to which the first wire 104A is connected) may be connected to a first organ 112 of the patient, and the second vagal trunk (e.g., to which the second wire 104B is connected) may be connected to a second organ 116. Additionally or alternatively, while the respective vagal trunks are shown as being connected to the corresponding organs of the patient as described, the vagal trunks to which the wires 104 are connected may be connected to the other organ (e.g., the first vagal trunk is connected to the second organ 116 and the second vagal trunk is connected to the first organ 112) or may be connected to different organs of the patient. In some examples, the first organ 112 may represent a liver of the patient, and the second organ 116 may represent a pancreas of the patient. In such examples, the blocking/stimulation signals provided by the wires 104 and the device 102 may be delivered to the liver and/or pancreas of the patient to mute a glycemic response of the patient as described herein.

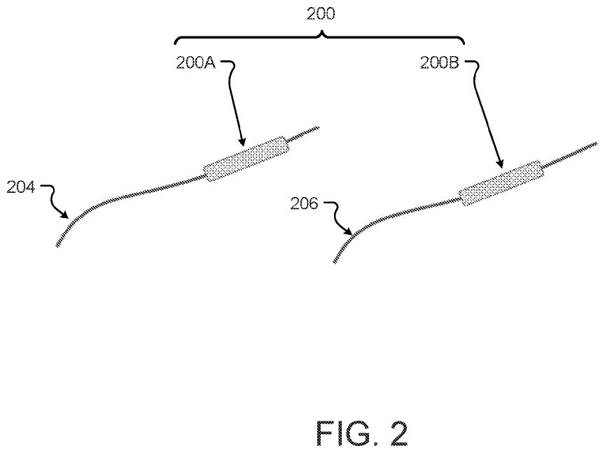

In some examples, the wires 104 may provide the electrical signals to the respective vagal trunks via electrodes that are connected to the vagal trunks (e.g., sutured in place, wrapped around the nerves of the vagal trunks, etc.). In some examples, the wires 104 may be referenced as cuff electrodes or may otherwise include the cuff electrodes (e.g., at an end of the wires 104 not connected or plugged into the device 102). Additionally or alternatively, while shown as physical wires that provide the connection between the device 102 and the one or more vagal trunks, the cuff electrodes may provide the electrical blocking and/or stimulation signals to the one or more vagal trunks wirelessly (e.g., with or without the device 102), as will be described in detail with respect to FIGS. 2-5.

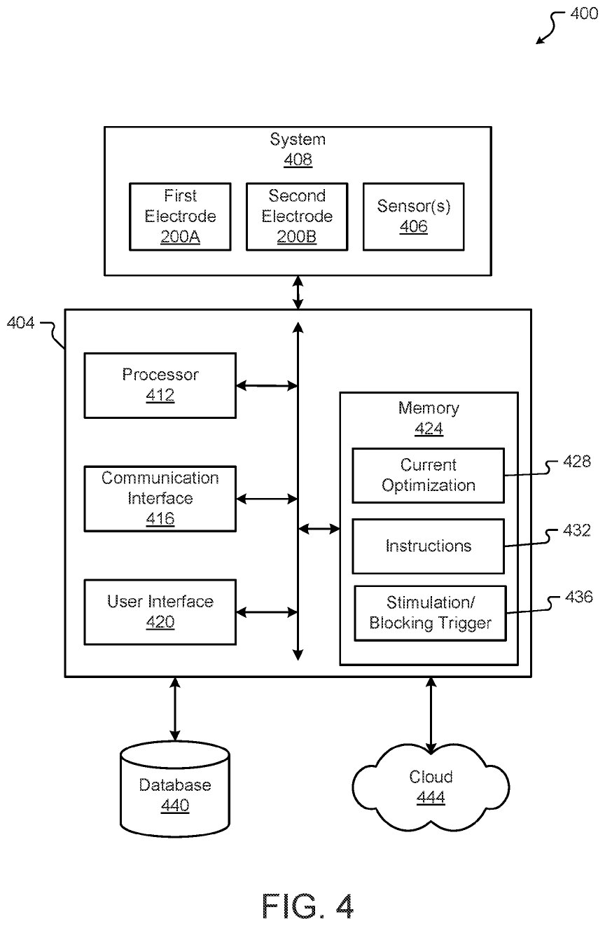

Additionally, the system 100 or components thereof may include one or more processors (e.g., one or more DSPs, general purpose microprocessors, graphics processing units, ASICs, FPGAs, or other equivalent integrated or discrete logic circuitry) shown and described in FIG. 4 that are programmed to carry out one or more aspects of the present disclosure. As an example, the processors may be provided in the device 102. In some examples, the device 102 may also include a memory to operate in connection with the processor. For example, the memory may provide instructions that are executed by processor(s) of the device to support functions of the device 102 described herein. In some examples, the one or more processors may be part of the device 102 or part of a control unit for the system 100 (e.g., where the control unit is in communication with the device 102 and/or other components of the system 100).

In some examples, the system 100 may also optionally include a glucose sensor 120 that communicates (e.g., wirelessly) with other components of the system 100 (e.g., the device 102, the one or more processors, etc.) to achieve better glycemic control in the patient. For example, the glucose sensor 120 may continuously monitor glucose levels of the patient, such that if the glucose sensor 120 determines glucose levels are outside a normal or desired range for the patient (e.g., glucose levels are too high or too low in the patient), the glucose sensor 120 may communicate that glucose levels are outside the normal or desired range to the device 102 (e.g., via the one or more processors) to signal for the device 102 to apply the stimulation/blocking therapy described herein to adjust glucose levels in the patient (e.g., mute the glycemic response to lower glucose levels in the patient, block insulin production in the patient as a possible technique to raise glucose levels in the patient, etc.).

The system 100 or components thereof may be used to carry out one or more aspects of any of the method described herein. The system 100 or similar systems may also be used for other purposes.

It will be appreciated that the human body has many vagal nerves and the stimulation and/or blocking therapies described herein may be applied to one or more vagal nerves, which may reside at any location of a patient (e.g., lumbar, thoracic, etc.). Further, a sequence of stimulations and/or blocking therapies may be applied to different nerves or portions of nerves. For example, a low frequency stimulation may be applied to a first nerve and a high frequency blockade may be applied to a second nerve.

Turning now to FIG. 2, electrodes 200 including a first electrode 200A and a second electrode 200B are shown. As shown in the illustrated embodiment, the first electrode 200A may be coupled to a first anatomical element 204—which may be, for example, a celiac branch of a vagal nerve—and the second electrode 200B may be coupled to a second anatomical element 206—which may be, for example, a hepatic branch of the vagal nerve. Each of the first electrode 200A and the second electrode 200B may be configured to deliver current provided by the device 102. In particular, electrodes 200A, 200B may provide the electrical interface between the wire(s) 104A, 104B and the anatomical elements 204, 206. It should be appreciated that one or both electrodes 200A, 200B may include one or many individual electrode elements (e.g., contact pads, exposed conductors, etc.).

The anatomical elements 204, 206 may comprise, for example, one or more nerves. In some embodiments, the first electrode 200A may be configured to apply a first current (e.g., having a first set of parameters) to the first anatomical element 204 and the second electrode 200B may be configured to apply a second current (e.g., having a second set of parameters) to the second anatomical element 206. The first current and the second current may have different parameters in some instances and may have the same parameters in other instances. For example, in some embodiments, the first current may comprise a low frequency stimulation and the second current may comprise a high frequency blockade.

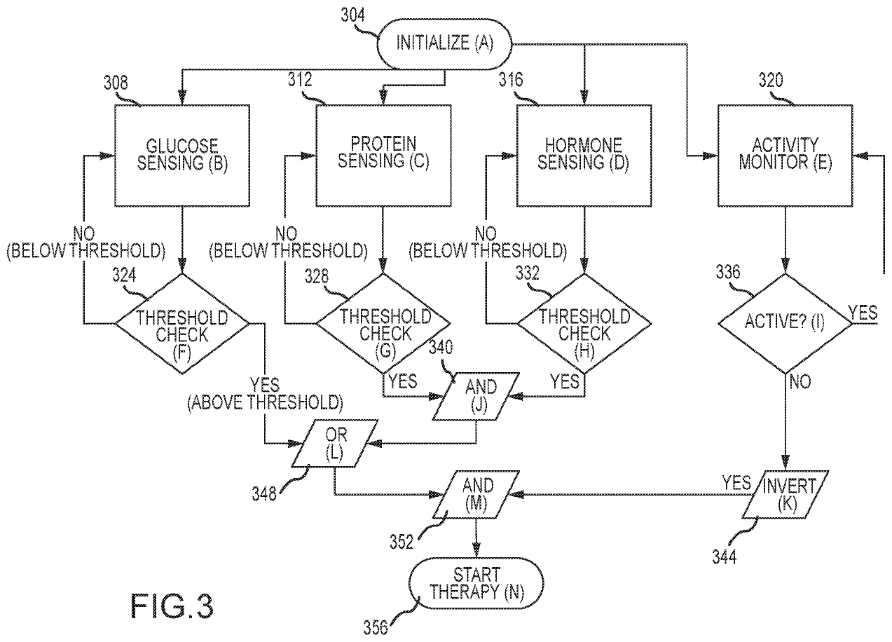

Turning to FIG. 3, additional details of a process for treating a diabetic condition with a closed-loop feedback system will be described in accordance with at least some embodiments of the present disclosure. The closed-loop feedback system may leverage feedback from one or more of a glucose sensor, a protein sensor, a hormone sensor, and an activity sensor. Said another way, the system 100 illustrated in FIG. 1 may be adjusted to include one or more of a glucose sensor, a protein sensor, a hormone sensor, and an activity sensor, each of which measure information about a patient and feedback the measurements to the device 102. Thus, while FIG. 1 does not necessarily illustrate a glucose sensor, a protein sensor, a hormone sensor, or an activity sensor, it should be appreciated that the system 100 may be configured to include one, some, or all of the above-mentioned sensors.

As an example, glucose levels of a patient may be measured with a continuous glucose sensor that is implanted in a patient. A non-continuous glucose sensor (e.g., a handheld glucose meter) may alternatively be used to measure glucose levels of a patient. It should be appreciated that more than one glucose sensor may be used to provide glucose measurement information (e.g., real-time glucose levels, A1C levels, average blood glucose levels, historical blood glucose levels, etc.). Thus, glucose sensing as described herein may be achieved using one or multiple glucose sensors/monitors.

A protein sensor may be used to measure protein levels of a patient. Alternatively, a patient may input meal information into a meal-tracking application that indirectly measures protein input for a patient. It should be appreciated that more than one protein s1ensor may be used to provide protein measurement information (e.g., real-time protein input levels, average protein input levels, historical protein input levels, etc.). Thus, protein sensing as described herein may be achieved using one or multiple protein sensors/monitors.

A hormone sensor may be used to measure various hormone levels of a patient. As an example, hormones of a patient that are measured with a hormone sensor include, without limitation, cortisol hormones, estrogen hormones, stress hormones, Luteinizing hormones, and the like. It should be appreciated that more than one hormone sensor may be used to provide hormone measurement information (e.g., real-time hormone levels, average hormone levels, historical hormone levels, etc.). Thus, hormone sensing as described herein may be achieved using one or multiple hormone sensors/monitors.

An activity sensor may be used to measure activity levels of a patient. Alternatively, a patient may input activity information into an application that indirectly measures activity input for a patient. It should be appreciated that more than one activity sensor may be used to provide activity measurement information (e.g., real-time activity levels, average activity levels, historical activity levels, etc.). Non-limiting examples of activity sensors include, accelerometers, gyroscopes, Global Positioning System (GPS) trackers, barometric pressure sensors, magnetic field sensors, electrodermal sensors, bioimpedance sensors, muscle activity or electromyographic sensors, etc. Thus, activity sensing as described herein may be achieved using one or multiple activity sensors/monitors.

FIG. 3 illustrates a closed-loop approach for the treatment of diabetes (e.g., type 2 diabetes) using the system described herein. The closed-loop approach may include a method that begins with an initialization of the system or components therein (step 304). To the extent that the system includes implantable components (e.g., an implantable device 102, implantable electrodes 200A, 200B, etc.) the initialization may occur prior to implantation in the patient or after the component(s) have been implanted (e.g., if the device 102 is remotely configurable). In the initialization step, the operation of the device 102 and thresholds associated with operating the closed-loop method may be set to default values or predetermined values that are calculated based on patient-specific information (e.g., height, weight, gender, etc.). At time of implant, initial parameters for the system are noted for baseline purposes. One, some, or all of these parameters may be adjustable. Examples of the parameters that may be set during the initialization include, without limitation:
A definition of a “normal” or threshold glucose range for the patient. For example, 100-110 mg/dL.
A definition of a protein baseline. Protein digestion will stimulate glucagon release independently of amino acid blood levels.
A definition of “normal” or threshold GLP1 levels (e.g., for hormone sensing)
A definition of a “resting activity level” or activity baseline (e.g., an accelerometer reading that is associated with patient resting). A heart rate sensor and/or other motion sensor can be utilized.

Following initialization, the method continues by glucose sensing (step 308), protein sensing (step 312), hormone sensing (step 316), and/or activity monitoring (step 320). Two or more sensing steps may be performed sequentially or simultaneously. It should also be appreciated that the measurements receiving in the sensing step may be received from an appropriate sensor of the system that is configured to measure patient glucose levels, protein levels, hormone levels, and activity levels.

In some embodiments, glucose sensing may occur continuously or periodically. As an example, Continuous Glucose Monitoring (CGM) may be used to check the patient's glucose level at regularly-defined intervals (e.g., every 5 minutes). It should be noted that the length of these intervals may be increased or decreased, depending upon patient need or care provider preferences. These periods may change based on time or state, such as a less frequent check at night/during sleep, or a more frequent check if the patient has indicated that a meal is occurring.

In some embodiments, protein sensing can be achieved using a portal amino acid sensor. Alternatively or additionally, the patient may indicate, via an application, that a meal of sufficient carbohydrate content (e.g., meeting or exceeding a determined carbohydrate threshold) has been ingested.

In some embodiments, hormone sensing may include GLP1 sensing with a chemical sensor. It should be appreciated that other or additional hormones may be monitored and sensed in this step.

As the various sensing activities occur, the method may continue by comparing measured values against threshold(s). In particular, the measured glucose levels may be compared against a predefined glucose threshold (step 324). As an example, if the measured glucose levels obtained from the CGM exceed a predetermined threshold (e.g., above 110 mg/dL or above 120 mg/dL), then the query of step 324 may be answered positively. If the query of step 324 is answered negatively, then the method returns to step 308 for further glucose sensing. If the query is answered positively, then the method proceeds to step 348 as will be described below.

The measured protein values may be compared to one or more appropriate thresholds (step 328). For example, this particular step may check to determine if ingested carbohydrates meet or exceed a predetermined carbohydrate threshold value. If the query is answered negatively, then the method returns to step 312. If the query is answered positively, then the method proceeds to step 340 as will be described below.

The measured hormone values may be compared to one or more appropriate hormone thresholds (step 332). As an example, GLP1 is a hormone that is secreted from the intestines during meal absorption. In step 332, measurements from a GLP1 sensor may be compared to an appropriate GLP1 threshold to determine if the measured values are greater than the GLP1 threshold. If the measured value does not exceed the threshold, then the method returns to step 316. If the measured value meets or exceeds the threshold, then the method proceeds to step 340 as will be described below.

The measured activity level(s) may be compared to one or more appropriate activity thresholds (step 336). As an example, measurements may be obtained from one or more activity sensors to determine if the patient is exercising or highly active. If the activity sensors indicate that the patient is currently involved in exercise or a high level of activity (e.g., heart rate is above a predetermined threshold, body temperature is above a predetermined threshold, motion is exceeding a predetermined threshold, and/or any other activity measurement indicates a likelihood of the patient not resting), then the query of step 336 is answered positively and the method returns to step 320. If the query of step 336 is answered negatively (e.g., the patient is determined to have an activity level below a predetermined threshold), then the method proceeds to step 344 as will be described below.

At step 340, an analysis is performed to determine if both threshold checks at steps 328 and 332 were answered positively. Specifically, this step is meant to reduce the sensitivity to either protein or hormone sensing individually. The results of the AND analysis performed in step 340 are then used to determine whether treatment should be performed on the basis of glucose sensing OR based on the combination of the protein sensing and hormone sensing (step 348). In particular, the analysis performed at step 348 represents that treatment will continue if glucose thresholds are satisfied OR if a combination of protein thresholds AND hormone thresholds are satisfied. As can be appreciated, if both conditions are satisfied, then the inquiry of step 348 is satisfied, meaning that the OR inquiry at step 348 will be satisfied based on the glucose threshold(s) being exceeded and/or based on the combination of the protein and hormone threshold(s) being exceeded.

At step 344, the binary signal (to the extent that a binary signal is being used) representing the output of the activity monitoring is inverted. The inversion process is performed since the other inquiries of steps 324, 328, and 332 trigger treatment in response to measured values exceeding a threshold whereas the inquiry of step 336 will trigger treatment only when measured activity values are below a predetermined activity threshold.

At step 352 the inverted output from step 344 is combined with the output of step 348. Step 352 represents the final AND statement that checks for a positive signal from a sensor or combinations of sensors, and permits treatment as long as one or more positive signals are received while also satisfying the condition that patient activity is below a predetermined threshold. If the AND statement at step 352 is satisfied, then the method continues by starting therapy (step 356). As will be described herein, the therapy applied to the patient may include one or both of applying an electrical stimulation and applying a nerve block (e.g., electrically or non-electrically/mechanically).

As can be seen in FIG. 4, a closed-loop system 400 may be used to implement the method depicted and described in FIG. 3. In particular, the system 400 may include a subsystem 408 having the electrode(s) 200A, 200B as well as one or more sensors 406. The sensors 406 of the system 408 may include any one or combination of sensors described in connection with the various sensing steps 308, 312, 316, 320. Inputs from the sensor(s) 406 may be provided to computing device 404, which may include functionality that determines whether and when to apply treatments to the patient via the electrode(s) 200A, 200B. Specifically, the computing device 404 may correspond to an example of device 102. The device 404 may be connected via wired connections or wirelessly with components of the system 408.

The system 400 is also shown to include a database 440 and/or a cloud or other network 444. Systems according to other embodiments of the present disclosure may comprise more or fewer components than the system 400. For example, the system 400 may not include one or more components of the computing device 404, the database 440, and/or the cloud 444.

In some embodiments, the computing device 404 is an example of device 102 or provides at least some functions described in connection with device 102. Illustratively, the computing device 404 includes a processor 412, a memory 424, a communication interface 416, and a user interface 420. Computing devices according to other embodiments of the present disclosure may comprise more or fewer components than the computing device 404.

The processor 412 of the computing device 404 may be any processor described herein or any similar processor. The processor 412 may be configured to execute instructions stored in the memory 424, which instructions may cause the processor 412 to carry out one or more computing steps utilizing or based on data received from the system 408, the database 440, and/or the cloud 444. Alternatively or additionally, the processor 412 may trigger patient treatment in the form of electrical stimulation and/or blocking based on information received from the sensor(s) 406, meaning that the processor 412 may deliver electrical signals or trigger electrical signals to be delivered to electrode(s) 200A, 200B.

The memory 424 may be or comprise RAM, DRAM, SDRAM, other solid-state memory, any memory described herein, or any other tangible, non-transitory memory for storing computer-readable data and/or instructions 432. The memory 424 may store information or data useful for completing, for example, any step of any method depicted and described herein. The memory 424 may store, for example, instructions and/or machine learning models that support functionality of the computing device 404. For instance, the memory 424 may store content (e.g., instructions 432 and/or machine learning models) that, when executed by the processor 412, enable the computing device 404 to receive inputs from one or more sensors 406, determine whether or not to trigger a therapeutic treatment (e.g., invoke electrical stimulation and/or blocking)(e.g., a trigger instruction set 436), invoke therapeutic treatment(s), and/or enable current optimization 428.

The trigger instruction set 436 may be used to process inputs received from sensor(s) 406 and determine whether to implement a treatment for a patient. The trigger instruction set 436, when executed by the processor 412, may also enable the device 404 to select an appropriate stimulation and/or blocking signal to apply to the patient via the electrode(s) 200A, 200B. For instance, the trigger instruction set 436 may select an appropriate waveform and duty cycle for application of either a stimulation signal or a blocking signal. In some embodiments, the trigger instruction set 436 may reference the current optimization 428 as part of determining which type of electrical signal(s) to apply to the patient and for how long such signals should be applied.

The current optimization 428 may correspond to a routine executed by the processor 412 to optimize current used in an electrical stimulation and/or nerve blocking. Optimization may be achieved by adjusting signal frequency, adjusting signal type (e.g., square wave, sinusoidal wave, triangle wave, etc.), adjusting duty cycle, adjusting treatment duration, etc. More specifically, the current optimization 428 may enable the processor 412 to determine one or more parameters of the current and/or a period of time to generate and apply the current. Blocking and stimulating waveforms may be delivered simultaneously, may be sequenced in time, or in other cases one or the other may be delivered independently based on the various states of the sensor inputs. As an example, one of the modalities might be delivered first, and then the second added only if the blood glucose muting effect fails to return the system to the desired state.

Content stored in the memory 424, if provided as in instruction 432, may, in some embodiments, be organized into one or more applications, modules, packages, layers, or engines. Alternatively or additionally, the memory 424 may store other types of content or data (e.g., machine learning models, artificial neural networks, deep neural networks, etc.) that can be processed by the processor 412 to carry out the various method and features described herein. Thus, although various contents of memory 424 may be described as instructions 432, it should be appreciated that functionality described herein can be achieved through use of instructions, algorithms, and/or machine learning models. The data, algorithms, and/or instructions may cause the processor 412 to manipulate data stored in the memory 424 and/or received from or via the system 408, the database 440, and/or the cloud 444.

The computing device 404 may also comprise a communication interface 416. The communication interface 416 may be used for receiving data (for example, data from sensor(s) 406) or other information from an external source (such as the system 408, the database 440, the cloud 444, and/or any other system or component not part of the system 400), and/or for transmitting instructions or signals to an external system or device (e.g., another computing device 404, the system 408, the database 440, the cloud 444, the electrode(s) 200A, 200B, and/or any other system or component not part of the system 400). The communication interface 416 may comprise one or more wired interfaces (e.g., a USB port, an Ethernet port, a Firewire port) and/or one or more wireless transceivers or interfaces (configured, for example, to transmit and/or receive information via one or more wireless communication protocols such as 404.11a/b/g/n, Bluetooth, NFC, ZigBee, and so forth). In some embodiments, the communication interface 416 may include a driver that facilitates generation/delivery of electrical stimulation and/or blocks via the electrode(s) 200A, 200B.

The computing device 404 may also comprise one or more user interfaces 420. The user interface 420 may be or comprise a keyboard, mouse, trackball, monitor, screen, touchscreen, lights, microphones, speakers, and/or any other device for receiving information from a user and/or for providing information to a user. The user interface 420 may be used, for example, to receive a user selection or other user input regarding any step of any method described herein. Notwithstanding the foregoing, any required input for any step of any method described herein may be generated automatically by the system 400 (e.g., by the processor 412 or another component of the system 400) or received by the system 400 from a source external to the system 400. In some embodiments, the user interface 420 may be useful to allow a surgeon or other user to modify instructions to be executed by the processor 412 according to one or more embodiments of the present disclosure, and/or to modify or adjust a setting of other information displayed on the user interface 420 or corresponding thereto.

Although the user interface 420 is shown as part of the computing device 404, in some embodiments, the computing device 404 may utilize a user interface 420 that is housed separately from one or more remaining components of the computing device 404. In some embodiments, the user interface 420 may be located proximate one or more other components of the computing device 404, while in other embodiments, the user interface 420 may be located remotely from one or more other components of the computer device 404.

The database 440 may store information such as patient data, results of a stimulation and/or blocking procedure, stimulation and/or blocking parameters, current parameters, electrode parameters, prior responses to stimulation in the patient, etc. The database 440 may be configured to provide any such information to the computing device 404 or to any other device of the system 400 or external to the system 400, whether directly or via the cloud 444. In some embodiments, the database 440 may be or comprise part of a hospital data storage system, a health information system (HIS), and/or another system for collecting, storing, managing, and/or transmitting electronic medical records.

The cloud 444 may be or represent the Internet or any other wide area network. The computing device 404 may be connected to the cloud 444 via the communication interface 416, using a wired connection, a wireless connection, or both. In some embodiments, the computing device 404 may communicate with the database 440 and/or an external device (e.g., a computing device) via the cloud 444.

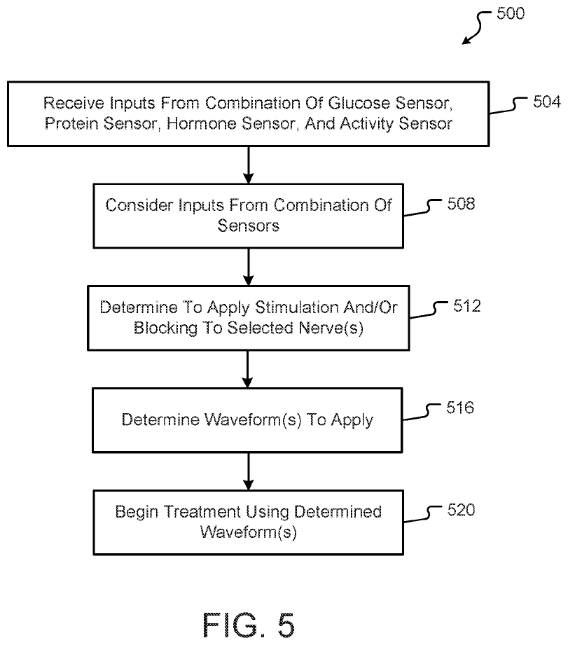

FIG. 5 illustrates a method 500 that may be used, for example, for implementing a closed-loop treatment program as described herein.

The method 500 (and/or one or more steps thereof) may be carried out or otherwise performed, for example, by at least one processor. The at least one processor may be the same as or similar to the processor(s) 412 of the computing device 404 described above. processor other than any processor described herein may also be used to execute the method 500.

The method 500 may begin when the processor 412 receives inputs from one, two, three, or more of a glucose sensor, protein sensor, hormone sensor, and activity sensor (step 504). It should be appreciated that the processor 412 may receive glucose readings from more than one glucose sensor, for example. Similarly, the processor 412 may receive protein readings from more than one protein sensor. The processor 412 may also receive hormone readings from more than one hormone sensor. The processor 412 may also receive activity readings from more than one activity sensor. The inputs received in step 504 may correspond to real-time readings from sensor(s) 406 and/or to historical readings obtained from a database 440, the cloud 444, or other remote data repositories.

The method 500 continues with the processor 412 utilizing the trigger instruction set 436 to process/consider the inputs received from the combination of sensors (step 508). Analysis of the measured inputs may result in the processor 412 determining whether or not to apply a stimulation and/or blocking signal to one or more selected nerves (step 512). In some embodiments, the determination may be made affirmatively in response to traversing the method depicted and described in connection with FIG. 3.

The method 500 then continues with the processor 412 determining which waveform(s) to apply as part of the treatment (step 516). This step may also include determining parameter(s) of the signal(s) to apply, when to start application of the selected signal(s), how long to continue application of the selected signal(s), inputs to consider as part of discontinuing application of the signal(s), etc.

The method 500 then continues with the processor 412 initiating treatment of the patient using signals having the determined parameter(s) (step 520). The method 500 is shown as being linear, but it should be appreciated that the method may be continuously repeated or updated. For instance, the method 500 may include a closed-loop and continuous process in which some or all steps of the method 500 are performed and re-performed until some predetermined condition is met. Examples of such predetermined conditions include: expiration of a timer, receiving a patient input to stop treatment, receiving a care provider's input to stop treatment, measuring some sensor input (e.g., glucose, protein, hormone, etc.) as falling below a predetermined value, measuring some sensor input (e.g., activity) as rising above a predetermined value, etc.

The present disclosure encompasses embodiments of the method 500 that comprise more or fewer steps than those described above, and/or one or more steps that are different than the steps described above.

As noted above, the present disclosure encompasses methods with fewer than all of the steps identified in FIG. 5 (and the corresponding description of the method 500), as well as methods that include additional steps beyond those identified in FIG. 5 (and the corresponding description of the method 500). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system, comprising:
a first sensor that measures a glycemic level of a patient;
a second sensor that measures at least one of a protein level of the patient, a hormone level of the patient, and an activity level of the patient;
a processor that receives inputs from the first sensor and inputs from the second sensor; and
memory comprising data that, when executed by the processor, enables the processor to:
analyze the inputs received from the first sensor and the second sensor;
determine, based on the analysis, that an electrical treatment is to be applied to the patient, wherein the electrical treatment comprises application of at least one electrical signal to a nervous system of the patient; and
cause the electrical treatment to be applied to the nervous system of the patient.

2. The system of claim 1, wherein the electrical treatment comprises application of a first electrical signal to a first portion of a nerve and application of a second electrical signal to a second portion of the nerve.

3. The system of claim 2, wherein first electrical signal comprises a low frequency stimulation of a celiac branch of the nerve and wherein the second electrical signal comprises a high frequency blockade of a hepatic branch of the nerve.

4. The system of claim 2, wherein the first electrical signal comprises a frequency of no more than about 5 kHz.

5. The system of claim 4, wherein the second electrical signal comprises a square wave having a frequency of between about 1-10 Hz.

6. The system of claim 2, wherein the second electrical signal comprises an ultralow frequency blockade of a hepatic branch of the nerve.

7. The system of claim 1, wherein the second sensor measures the protein level of the patient, the system further comprising:
a third sensor that measures the hormone level of the patient.

8. The system of claim 7, wherein the memory further comprises data that, when executed by the processor, enables the processor to analyze the inputs received from the first sensor, the second sensor, and the third sensor and further enables the processor to determine, based on the analysis, that the electrical treatment is to be applied to the patient.

9. The system of claim 8, wherein the electrical treatment is applied when the following conditions are met:
(i) the measured glycemic level exceeds a predetermined glycemic threshold OR (ii) the measured protein level exceeds a predetermined protein threshold AND the measured hormone level exceeds a predetermined hormone threshold.

10. The system of claim 9, wherein the electrical treatment is applied when the measured activity level of the patient is below a predetermined activity threshold.

11. The system of claim 1, wherein the second sensor comprises an activity sensor that measures the activity level of the patient.

12. The system of claim 11, wherein the activity sensor comprises at least one of a heart rate sensor, an accelerometer, a gyroscope, and a motion sensor.

13. The system of claim 1, wherein first sensor comprises a continuous glucose monitor.

14. A device comprising:
a processor that receives a first input from a first sensor and a second input from a second sensor, wherein the first input describes a glycemic level of a patient, and wherein the second input describes at least one of a hormone level, a protein level, and an activity level of the patient; and
memory comprising data that, when executed by the processor, enables the processor to;
analyze the first input and the second input;
determine, based on the analysis, that a treatment is to be applied to the patient, wherein the treatment comprises application of at least one electrical signal to a nervous system of the patient; and
cause the treatment to be applied to the patient.

15. The device of claim 14, wherein the treatment comprises application of a first electrical signal to a first portion of a nerve and application of a second electrical signal to a second portion of the nerve, wherein first electrical signal comprises a low frequency stimulation of a celiac branch of the nerve, and wherein the second electrical signal comprises a high frequency blockade of a hepatic branch of the nerve.

16. The device of claim 14, wherein the processor receives a third input from a third sensor, wherein the second input describes the hormone level, wherein the third input describes the protein level, and wherein the memory further comprises data that, when executed by the processor, enables the processor to analyze the third input along with the first input and the second input, then determine, based on the analysis, that the treatment is to be applied to the patient.

17. The device of claim 16, wherein the processor receives a fourth input from a fourth sensor, wherein the fourth input describes the activity level, and wherein the memory further comprises data that, when executed by the processor, enables the processor to analyze the fourth input along with the first input, the second input, and the third input, then determine, based on the analysis, that the treatment is to be applied to the patient.

18. The device of claim 17, wherein the fourth sensor comprises at least one of a heart rate sensor, an accelerometer, a gyroscope, and a motion sensor.

19. A closed-loop system for providing therapy to a patient, the system comprising:
a plurality of sensors, wherein the plurality of sensors measure two or more of a glycemic level of the patient, a hormone level of the patient, a protein level of the patient, and an activity level of the patient;
a processor; and
memory comprising data that, when executed by the processor, enables the processor to;
receive inputs from the plurality of sensors;
analyze the inputs received from the plurality of sensors;
determine, based on the analysis, that a treatment is to be applied to the patient, wherein the treatment comprises application of at least one electrical signal to a nervous system of the patient; and
cause the treatment to be applied to the patient.

20. The closed-loop system of claim 19, further comprising:
a first electrode that delivers a first electrical signal to a first portion of a nerve; and
a second electrode that delivers a second electrical signal to a second portion of the nerve.

21. The closed-loop system of claim 19, wherein the processor and the memory are included in an implantable pulse generator.
